(19) United States
(12) Patent Application Publication
(10) Pub. No.: US 2010/0076092 A1
(43) Pub. Date: Mar. 25, 2010

(54) LIPID-DERIVED NANOPARTICLES FOR BRAIN-TARGETED DRUG DELIVERY

(76) Inventors: Jayanth Panyam, Novi, MI (US); Mahesh D. Chavanpatil, Detroit, MI (US)

Correspondence Address: C. Rachal Winger;c/o OCIP Group, 1900 Main Street, Suite 600, Irvine, CA, 92614-7319, US

(21) Appl. No.: 12/377,599

(22) PCT Filed: Aug. 21, 2007
(86) PCT No.: PCT/US07/76389

§ 371 Date: Dec. 2, 2009

Related U.S. Application Data
(60) Provisional application No. 60/839,467, filed on Aug. 22, 2006.

(51) Int. Cl. A61K 47/14

(52) U.S. Cl. 514/784

(57) ABSTRACT

Disclosed herein are compositions and methods useful for the delivery of compounds across the blood brain barrier. Nanoparticles comprising lipids, such as brain lipids, are used to encapsulate drugs and to facilitate drug delivery to the brain.

### FIELD OF THE INVENTION

Compositions and methods of delivering compounds through the blood brain barrier into the brain are provided.

### BACKGROUND

Movement of compounds from circulating blood into the brain is tightly controlled by the brain capillary endothelium, which forms what is known as the blood-brain barrier (BBB). The BBB is formed by two primary elements (Bauer, B. et al., Experimental Biology and Medicine, 230:118-127, 2005). The first element is a diffusional barrier, formed by tight junctions between brain capillary endothelial cells. Tight junctions prevent the diffusional penetration of hydrophilic and/or large molecular weight molecules (Pardridge, W. M., Physiol Rev, 63:1481-1535, 1983, Levin, V. A., J Med Chem, 23:682-684, 1980). Low rate of endocytosis in the brain capillary endothelial cells provides further resistance to drug transport across the endothelial cells (Pardridge, W. M. et al., J Pharmacol Exp Ther, 253:884-891, 1990). The second element is a more selective barrier that is driven by membrane-bound drug efflux transporters like P-gp (Fromm, M. F., Trends Pharmacol Sci, 25:423-429, 2004). Together, the two elements protect the brain from potentially toxic foreign chemicals.

The barrier function of the endothelium also denies entry to therapeutic drugs that have the brain as their site of action. In general, only small molecular weight, lipophilic drugs, such as general anesthetics, analgesics and other central nervous system drugs are able to freely diffuse into the brain. Alternatively, a limited number of compounds such as growth factors are able to cross the BBB via specific receptors.

However, it is estimated that up to 98% of the newly developed small molecules will not cross the BBB (Pardridge, W. M., Mol. Interv., 3: 90-105, 2003). As a consequence, many diseases that afflict the brain, including cancer (Kemper, E. M. et al., Cancer Treatment Reviews, 30: 415-423, 2004), viral (Kim, R. B. et al., J Clin Invest, 101:289-294, 1998) and bacterial infections (Imbert, F. et al., Drug Metab Dispos, 31:319-325, 2003), as well as refractory epilepsy (Loscher, W. and Potschka, H., J Pharmacol Exp Ther, 301:7-14, 2002), have been extremely difficult to treat.

There is a pronounced need in the art for new mechanisms of delivering drugs to the brain.

### SUMMARY OF THE INVENTION

Embodiments of the present invention relate to nanoparticle compositions comprising brain lipids, supplemental lipids, polyethylene glycol-conjugated lipids, and drugs or therapeutic compounds.

Further embodiments of the invention relate to compositions wherein the brain lipid is a phospholipid, such as phosphatidylethanolamine, phosphatidylserine, phosphatidylcholine, phosphatidic acid, and phosphatidylinositol.

Still further embodiments of the present invention relate to compositions wherein the supplemental lipid is a long chain fatty acid, such as stearic acid, palmitic acid, linolic acid, or linoleic acid.

Additional embodiments of the present invention relate to compositions wherein the polyethylene glycol-conjugated lipid is distearoylphosphatidylethanolamine-polyethylene glycol.

Other embodiments of the present invention relate to compositions wherein the drug or therapeutic compound is a drug targeted toward the central nervous system.

Still other embodiments of the present invention relate to compositions wherein the drug or therapeutic compound is a hydrophobic drug, an anti-cancer drug, a drug for the treatment of epilepsy, a drug for the treatment of brain injury, a drug for the treatment of amylotrophic lateral sclerosis, a drug for the treatment of bacterial infections, or a drug for the treatment of viral infections.

Embodiments of the present invention relate to the use of nanoparticles to deliver a drug or therapeutic compound to the brain of an individual, comprising a nanoparticle composition and administering the composition to the individual.

### BRIEF DESCRIPTION OF THE FIGURES

FIG. 1 shows the effect of brain-derived lipid on brain delivery of BMS 182874 (BMS).

FIG. 2 shows the effect of brain-derived lipid on brain delivery of 6-coumarin.

FIG. 3 shows a plot relating sonication energy (SE), sonication time (ST), homogenization speed (HS), and homogenization time (HT) to effective diameter (ED).

FIG. 4 shows an interaction effects plot for effective diameter (ED).

FIGS. 5A and B show AFM images of nanoparticles from Experiment 6 at lower (A) and higher magnifications (B).

FIGS. 6A and B show AFM images of nanoparticles from Experiment 17 at lower (A) and higher magnifications (B).

FIG. 7 shows a plot relating sonication energy (SE), sonication time (ST), homogenization speed (HS), and homogenization time (HT) to zeta potential (ZP).

FIG. 8 shows an interaction effects plot for zeta potential (ZP).

FIG. 9 shows a plot relating sonication energy (SE), sonication time (ST), homogenization speed (HS), and homogenization time (HT) to polydispersity (PD).

FIG. 10 shows an interaction effects plot for polydispersity (PD).

FIG. 11 shows the effect of storage on particle size of nanoparticles from Experiment 10 and Experiment 13.

FIG. 12 shows AFM images of nanoparticles from Experiment 10 stored at 4° C. for 48 hours at lower (A) and higher magnifications (B).

FIG. 13 shows AFM images of nanoparticles from Experiment 13 stored at 4° C. for 48 hours at lower (A) and higher magnifications (B).

FIG. 14 shows AFM images of nanoparticles from Experiment 10 at 0 hour at lower (A) and higher magnifications (B).

FIG. 15 shows AFM images of nanoparticles from Experiment 13 at 0 hour at lower (A) and higher magnifications (B).

### DETAILED DESCRIPTION

Unless defined otherwise, technical and scientific terms used herein have the same meaning as commonly understood by one of ordinary skill in the art to which this invention belongs. The invention disclosed herein includes compositions and methods relating to novel formulations of nanoparticles comprising brain-derived lipids. Such lipids enhance drug delivery to the brain, and may be useful for administering drugs to treat diseases including but not limited to epilepsy, Parkinson's disease, Alzheimer's disease, stroke and brain injury, brain cancer, HIV infection of the brain, and amyotrophic lateral sclerosis.

For the purpose of this disclosure, nanoparticles are defined as colloidal particles of sub-micron size of 10-1000 nanometers, such as 30-500 nanometers, or 50-350 nanometers, with the drug of interest entrapped into the matrix (Panyam, J. and Labhasetwar, V., Adv Drug Deliv Rev, 55: 329-347, 2003). Nanoparticles may be referred to as nanospheres, nanogels, nanocapsules, and micelles. The sub-micron size of nanoparticles has the advantages that they allow for cellular and tissue uptake, and they have the ability to pass through fine capillaries. Use of biodegradable materials in nanoparticle formulation may allow for sustained drug release at the target site over a period of weeks after injection (Panyam, J. and Labhasetwar, V., Adv Drug Deliv Rev, 55: 329-347, 2003).

The inventive nanoparticles may comprise “brain lipids” that enhance the delivery of compounds across the blood-brain barrier. The term “brain lipids” refers to any of a number of generally non-polar compounds that are found in the cell membranes of mammalian brains. In general, brain lipids comprise phospholipids, but may also comprise compounds containing long-chain fatty acids and their derivatives, as well as linked isoprenoid units. The fatty acids in lipids may be either esterified to the trihydroxy alcohol glycerol; or present as amides of sphingosine, a long-chain dihydroxyamine. The isoprenoids may be made up of branched-chain units and may include sterols, such as cholesterol. Examples of brain lipids include but are by no means limited to phosphatidylethanolamine, phosphatidylserine, phosphatidylcholine, phosphatidic acid, and phosphatidylinositol. Avanti Polar Lipids, for example, supplies a brain lipid extract that has the composition shown in Table 1 (Avanti catalog No. 141101).

TABLE 1
Composition of brain-derived lipids

| Component | Percent/Wt. |
|---|---|
| Phosphatidylethanolamine | 33.1 |
| Phosphatidylserine | 18.5 |
| Phosphatidylcholine | 12.6 |
| Phosphatidic Acid | 0.8 |
| Phosphatidylinositol | 4.1 |
| Other | 30.9 |

Alternatively, the lipid component mixture of the nanoparticles may be made in vitro by mixing individual purified lipids rather than extracting lipids from brain cell membranes. Making lipid mixtures in this manner allows for greater flexibility in the nanoparticle composition.

The inventive nanoparticles generally further comprise other supplemental lipids. These supplemental lipids are generally non-brain lipids such as long chain saturated or unsaturated fatty acids. For example, stearic acid, linolic acid, palmitic acid, and linoleic acid may be included as supplemental lipids in nanoparticles. If a brain extract is used, additional lecithin (phosphatidylcholine) may be added to the nanoparticle mixture.

Additionally, other lipids that have desirable properties, such as those that promote stability or extended circulation times, or allow the nanoparticles to avoid detection by a recipient's immune system, may be included in the inventive nanoparticles. For example, lipids that are conjugated to polyethylene glycol (PEG) may be used for this purpose. A PEG-conjugated lipid compound that may be useful is distearoylphosphatidylethanolamine-PEG (DSPE-PEG).

The inventive nanoparticles may be used to encapsulate a wide range of drugs or therapeutic compounds, including but not limited to hydrophobic drugs, anti-cancer drugs, drugs for the treatment of epilepsy, brain injury, amylotrophic lateral sclerosis, bacterial infections, viral infections, or any other drug that may be used to act on the central nervous system or otherwise alter the psychopharmacology of an individual. For example, the compound BMS 182874, which is an endothelin-1 antagonist that has potential applications in stroke but generally exhibits poor penetration into the brain, may be encapsulated in nanoparticles. In addition, other drugs, such as clonidine or taxol, may be encapsulated.

The nanoparticles are generally made by dissolving one or more drugs, brain lipids, and other components dissolved in chloroform or other suitable organic solvent. The chloroform may then be evaporated under nitrogen or argon to produce a clear lipid phase. A pre-dispersion of lipid in distilled water may then be prepared by magnetic stirring. This dispersion may then be passed through a high-pressure homogenizer or be sonicated to produce the nanoparticles. If desired, the size of the nanoparticles may be measured by dynamic light scattering, or any other suitable method.

The nanoparticles disclosed herein may be used to treat a variety of conditions that benefit from the introduction of particular compounds to the brain. The nanoparticles may be introduced by any number of suitable means, but in preferred embodiments, they are introduced to an individual intravenously or intraperitoneally.

Without being bound by a specific mechanism, nanoparticles disclosed herein containing lipids, such as nanoparticles containing brain-derived lipids, may be transported into the brain through specific receptors for these lipids. The inventors'studies indicate that these nanoparticles efficiently encapsulate drugs. As shown in Example 2 below, studies in mice indicate that these nanoparticles increase the brain delivery of encapsulated drugs following systemic administration. Increased and targeted delivery of drugs to the brain enhances the therapeutic efficacy of drugs that have the central nervous system as the site of action.

Physical properties such as particle size, polydispersity, and zeta potential are important determinants of biological performance of nanoparticles. Formulation process parameters can greatly influence the physical properties of nanoparticles. Example 1 herein utilized a 2⁴ factorial design to determine the effect of processing conditions such as sonication energy, sonication time, homogenization energy, and homogenization time on particle size, polydispersity, and zeta potential of a phospholipid nanoparticle formulation. The main effects of the factors and interactions between them were modeled using factorial analysis.

The results indicate that sonication energy, sonication time, and homogenization speed were important determinants of nanoparticle size. It was observed that an increase in homogenization speed resulted in an increase in particle size; however, elimination of the homogenization step resulted in nanoparticles that were more irregular in shape and in the formation of aggregates as observed by atomic force microscopy (AFM). Zeta potential was affected only marginally by any of the process parameters. Sonication energy was the biggest determinant of nanoparticle polydispersity. AFM studies suggest that although two different energy inputs may result in similar particle size, nanoparticles prepared with lower energy input are more stable against aggregation upon storage. Factorial design in conjunction with AFM was successfully used to predict the effect of various process parameters on physical properties of phospholipid nanoparticles.

Particle size and zeta potential are often used to characterize nanoparticles, because these measurements facilitate the understanding of the dispersion and aggregation processes (Heurtault et al., Biomaterials, 24:4283-4300, 2003). Further, particle size and zeta potential affect the biologic handling of nanoparticles (Desai et al., Pharm. Res. 14, 1568-1573, 1997). Many factors including nanoparticle composition and processing conditions influence particle size and zeta potential (Heurtault et al., Biomaterials, 24:4283-4300, 2003). The classical approach used to optimize nanoparticle characteristics is to vary one factor while keeping the others constant. This approach generally leads to a large number of experiments and is unlikely to reveal the presence of factor interactions. In preparing the present nanoparticles, factorial design was used to enable all factors to be varied simultaneously, allowing quantification of the effects caused by independent variables and interactions between them (Bozkir and Saka, Farmaco 60:840-846, 2005).

Particle size can influence the biopharmaceutical properties of a formulation by affecting the drug release kinetics. Smaller particles have a larger free surface area exposed to the leaching medium, resulting in a faster release of the incorporated therapeutic agent. Smaller size also leads to a shorter average diffusion path of matrix-entrapped drug molecules (Chorny et al., J. Control. Release 83, 389-400, 2002). Particle size can also influence the biodistribution and tissue uptake of particles. For example, it was shown that penetration through the mucus and submucosal layers and gastrointestinal absorption depends on particle size (Desai et al., Pharm. Res. 13, 1838-1845.1996; Hillyer and Albrecht, J. Pharm. Sci. 90, 1927-1936, 2001; Lai et al., Proc. Natl. Acad. Sci. USA. 104, 1482-1487. 2007).

Experiments described in Example 1 utilized factorial design to determine the effect of formulation process parameters on particle size, polydispersity, and zeta potential of the phospholipid-containing lipid nanoparticle formulations of the invention. Such lipid nanoparticles provide industrial applicability and offer the possibility of controlled drug release and drug targeting, increased drug stability, high drug payload, easy scalability, and sterilization (Mehnert and Mader, Adv. Drug Deliv. Rev. 47:165-196, 2001; Wang et al., Eur. J. Pharm. Biopharm. 54:285-290, 2002; Wissing et al., Adv. Drug Deliv. Rev. 56:1257-1272; 2004).

The Examples shown below are included for purposes of illustration only, and are not intended to limit the scope of the range of techniques and protocols in which the nanoparticles of the present invention may find utility, as will be appreciated by one of skill in the art.

### Example 1

### Phospholipid Nanoparticles

Lecithin (granular), stearic acid, chloroform, and polyethylenimine (PEI) were obtained from Sigma-Aldrich, St Louis, Mo. DSPE-PEG 2000[1,2-Distearoyl-sn-Glycero-3-Phosphoethanolamine-N-[Amino (Polyethylene Glycol) 2000] (Ammonium Salt)] and phospholipid mixture (in the form of polar brain extract) were obtained from Avanti Polar Lipids, Alabaster, Ala.

Nanoparticles were produced using sonication and homogenization in sequence. The two methods use different approaches to achieve size reduction. Sonication causes cavitation, the formation and violent collapse of microscopic bubbles. The collapse of thousands of cavitation bubbles releases tremendous energy in the cavitation field. The emulsion droplets/particles within the cavitation field are size-reduced. Homogenization results in the “forcing” of emulsion droplets/particles through a microscopic orifice that results in shearing and size reduction. Although both methods have been used individually in the production of nanoparticles (Mehnert and Mader, Adv. Drug Deliv. Rev. 47:165-196, 2001), use of any one method at high intensity may be detrimental to the therapeutic agent (Cegnar et al., Eur. J. Pharm. Sci. 22:357-364, 2004) incorporated in the formulation or to the formulation itself (Reich, G., Eur. J. Pharm. Biopharm. 45:165-171, 1998). For this reason, the use of sub-maximal levels of both methods was taken into account to achieve the desired nanoparticle properties.

The influence of processing conditions including sonication energy, sonication time, homogenization energy, and homogenization time on nanoparticle size, polydispersity, and zeta potential was evaluated using a two-level factorial design. The four independent variables, i.e. sonication energy (X₁), sonication time (X₂), homogenization energy (X₃), and homogenization time (X₄) were taken at two levels, high and low, which were represented by transformed values of +1 and −1 (Table 1). This design offers the possibility of investigating four independent variables at two levels after performing only sixteen experiments (Table 2).

Fitting a multiple regression model to the 2⁴ factorial design gave a predictor equation, which is a first-order polynomial having the form:

Y=b₀+b₁X₁+b₂X₂+b₃X₃+b₄X₄+b₁₂X₁X₂+b₁₃X₁X₃+b₁₄X₁X₄b₂₃X₂X₃+b₂₄X₂X₄+b₃₄X₃X₄+b₁₂₃X₁X₂X₃+b₁₂₄X₁X₂X₄+b₂₃₄X₂X₃X₄+b₁₃₄X₁X₃X₄+b₁₂₃₄X₁X₂X₃X₄

Where Y is the level of a given response (dependant variable), b terms are the regression coefficients for the first order polynomial and X is the level of the independent variable.

Data was analyzed using MINITAB™ software. Statistical comparisons were made using ANOVA. The coefficients were considered significant at P<0.05.

Phospholipid nanoparticles were prepared as follows. Lecithin (23 mg), stearic acid (30 mg), DSPE-PEG 2000 (9 mg), and phospholipid mixture (6 mg) were dissolved in 10 ml chloroform, and chloroform was evaporated using a rotary vacuum evaporator (Buchi Rotavapor R-1200, Buchi Labortechnik AG, Switzerland) to produce a lipid phase. A predispersion of the lipid phase in 5 ml of deionized water was prepared by sonication (Model 3000, Misonix, Farmingdale, N.Y.) for variable time intervals and at different sonication energies. The dispersion was then passed through a hand-held homogenizer (Tissuemiser, Fisher Scientific, Chicago, Ill.) at variable homogenization speeds and for variable times. Nanoparticles formed were recovered by ultracentrifugation (L7-165 Ultracentrifuge, Beckman, Palo Alta, Calif.) at 30,000 rpm for 30 minutes, washed twice with deionized water, resuspended in deionized water and lyophilized (Freeze Dry System/Freezone 4.5, Labconco, Kansas City, Mo.).

Particle size and polydispersity of nanoparticles were determined by dynamic light scattering. About 1 mg of nanoparticles was dispersed in 1 ml of deionized water by sonication at 3 W for 60 seconds. Sixty μl of this dispersion was diluted to 3 ml with deionized water and then subjected to effective diameter and polydispersity measurements using Brookhaven 90Plus equipment (Brookhaven Instruments, Holtsville, N.Y.).

Particle morphology was evaluated using Atomic Force Microscopy (AFM) (Nanoscope III, Digital Instruments/VEECO) in the tapping mode. For AFM, silicon tapping tips (TESP, VEECO) were used with a nominal tip radius less than 10 nm as provided by the manufacturer. One mg of nanoparticles was dispersed in 1 ml of deionized water by sonication at 3 W for 60 seconds. One hundred μl of this dispersion was diluted to 1 ml with deionized water and a droplet of the nanoparticle dispersion was placed on a PEI—coated glass cover slip and air dried. Nanoparticles were then imaged with an AFM E scanner (maximum scan area=14.2×14.2 μm²). The scan rate was 1 Hz and the integral and proportional gains were approximately 0.4 and 0.7 respectively. Height images were plane-fit in the fast scan direction with no additional image filtering.

Zeta potential was determined using Electrophoretic Light Scattering using Bróokhaven 90Plus equipment. One mg of nanoparticles was dispersed in 1 ml of deionized water by sonication at 3 W for 60 seconds. One hundred μl of this dispersion was diluted to 2 ml with deionized water and subjected to zeta potential measurement.

To study the effect of storage on the particle size and morphology of nanoparticles, two formulations (Experiment 10 and 13; Table 2) were dispersed in phosphate buffered saline (pH 7.4, 0.15 M) and stored at 4° C., 25° C., and 37° C. Particle size was measured at 0 hour, 1 hour, 24 hours, 48 hours, and 168 hours. Before every measurement, nanoparticle suspension was dispersed using a low-speed shaker (Labquake Rotisserie, Barnstead Thermolyne, Dubuque, Iowa). Nanoparticle morphology was determined using AFM before and after storage at 4° C. for 48 hours.

This Example demonstrates the effect of sonication energy, sonication time, homogenization energy, and homogenization time on particle size, polydispersity, and zeta potential. Both main effects and interaction effects were considered. Main effects represent the average result of changing one factor at a time from its low to high value. An interaction between factors occurred when the effect of one factor was dependent upon a second factor. Interaction plots were used to compare the relative strength of the effects across factors.

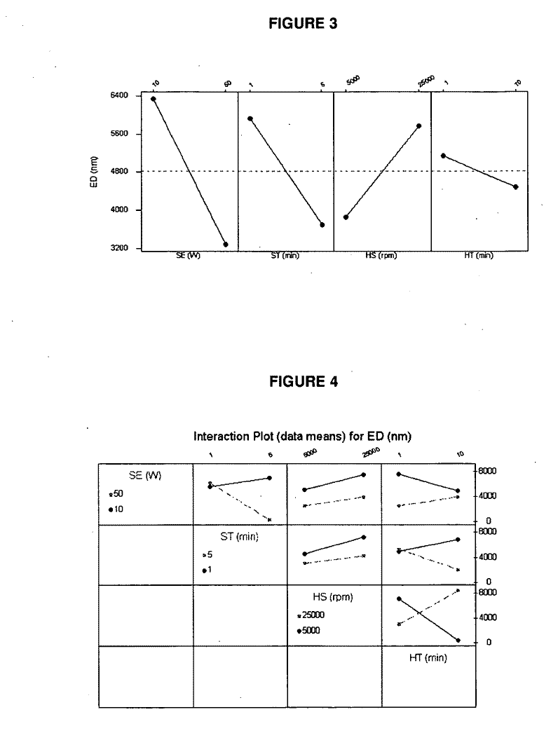

FIG. 3 shows that increasing either the sonication energy or the sonication time resulted in a significant decrease in particle size (P<0.05; Table 3). This finding is similar to those reported by Tang et al., Int. J. Pharm. 265, 103-114 (2003) and Mainardes and Evangelista, Int. J. Pharm. 290, 137-144 (2005). A longer duration of sonication may help disperse the polymeric organic phase as nanodroplets of smaller size and achieve a narrow size distribution profile. Increasing the homogenization time resulted in a slight decrease in particle size (not significant at P<0.05; Table 3).

Increasing homogenization speed resulted in a marginal increase in particle size, which was however not statistically significant. Mao et al. Effect of WOW process parameters on morphology and burst release of FITC-dextran loaded PLGA microspheres. Int. J. Pharm. in press (2006) observed a similar effect of homogenization speed on nanoparticle size. It was suggested the increase in particle size with increased homogenization speed could be due to particle coalescence which occurs due to high kinetic energy of particles (Muller et al., Eur. J. Pharm. Biopharm. 50, 161-177, 2000). From FIG. 4, it can be seen that there is a significant interaction between sonication energy and sonication time, sonication time and homogenization time, and homogenization speed and homogenization time in influencing the particle size (P<0.05; Table 3). On the other hand, there appears to be no interaction between sonication energy and homogenization speed or sonication energy and homogenization time or sonication time and homogenization speed.

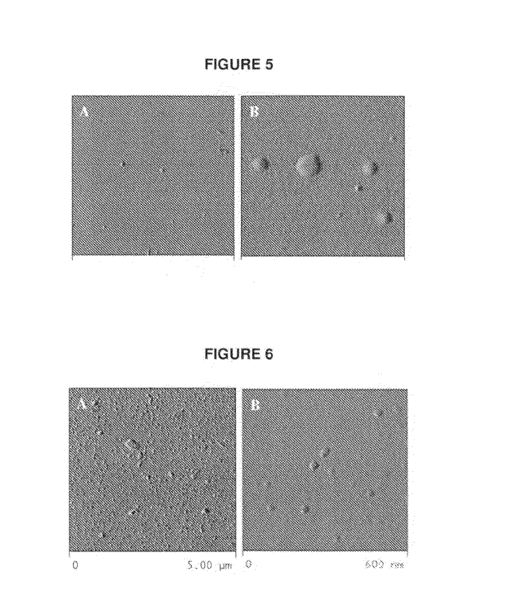

Because an increase in homogenization speed resulted in an increase in particle size, eliminating the homogenization step in nanoparticle production was an option. However, the use of sonication alone in nanoparticle formulation resulted in nanoparticles that were more irregular in shape and in the formation of aggregates. AFM images of nanoparticles prepared with homogenization (Experiment 6; Table 2; FIGS. 5A and B) and without (Experiment 17; Table 4; FIGS. 6A and B) demonstrate that homogenization is important to maintain the spherical morphology of nanoparticles and to eliminate the presence of aggregates.

This Example suggests that although an increase in homogenization speed results in an increase in particle size, low-speed homogenization is important to improve nanoparticle yield and to obtain spherical nanoparticles. It is important to note that dynamic light scattering provided larger particle size than AFM. Without being bound by a particular mechanism, this could be attributed to the following factors: (1) dynamic light scattering computes the hydrodynamic diameter while AFM provides the size for nanoparticles in “dry” state, and (2) dynamic light scattering computes z-average particle size while AFM provides number average particle size. The zeta potential of nanoparticles influences their dispersion stability.

Higher zeta potential values, both positive and negative, tend to stabilize particle suspensions because of electrostatic repulsion between particles with same electrical charge preventing aggregation (Vandervoort and Ludwig, Int. J. Pharm. 238, 77-92, 2002). The zeta potential also plays an important role in recognition by the reticuloendothelial system. The zeta potential can be related to the thickness of the adsorbed layer of blood components (opsonins) onto the particles that can enhance phagocytosis by macrophages (McCarron et al., Int. J. Pharm; 193, 37-47.1999). Particles with lower charges experience reduced macrophage uptake and increased circulation times (Feng and Huang, J. Control. Release. 71, 53-69, 2001).

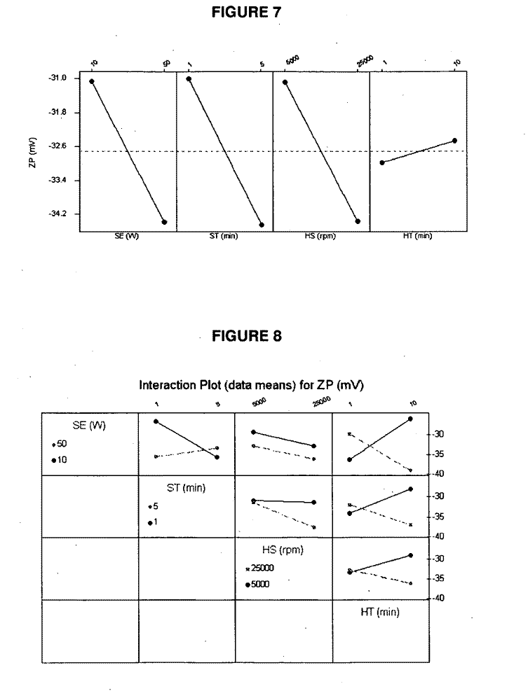

FIG. 7 shows that increasing the sonication energy, sonication time, and homogenization speed resulted in a marginal but statistically significant (P<0.05; Table 5) decrease in the zeta potential. Similar to that observed for particle size, homogenization time appeared to have no impact on zeta potential. FIG. 8 shows that there was an interaction between sonication energy and sonication time, sonication energy and homogenization time, sonication time and homogenization time, and homogenization speed and homogenization time in influencing zeta potential. On the other hand, there appeared to be no interaction between sonication energy and homogenization speed or sonication time and homogenization speed.

Although the above mentioned interaction effects are statistically significant (Table 5), only a small change (˜3-4 mV) in zeta potential was observed for the different formulations. This suggests that processing parameters play only a small role in determining the zeta potential of nanoparticles. Other studies suggest that the formulation itself (i.e., the excipients used in the formulation) could influence zeta potential to a greater extent (McCarron et al., Int. J. Pharm. 193, 37-47.1999; Vandervoort and Ludwig, Int. J. Pharm. 238, 77-92, 2002; Yang, Tumor necrosis factor alpha blocking peptide loaded PEG-PLGA nanoparticles: Preparation and in vitro evaluation. Int. J. Pharm. In press, 2006).

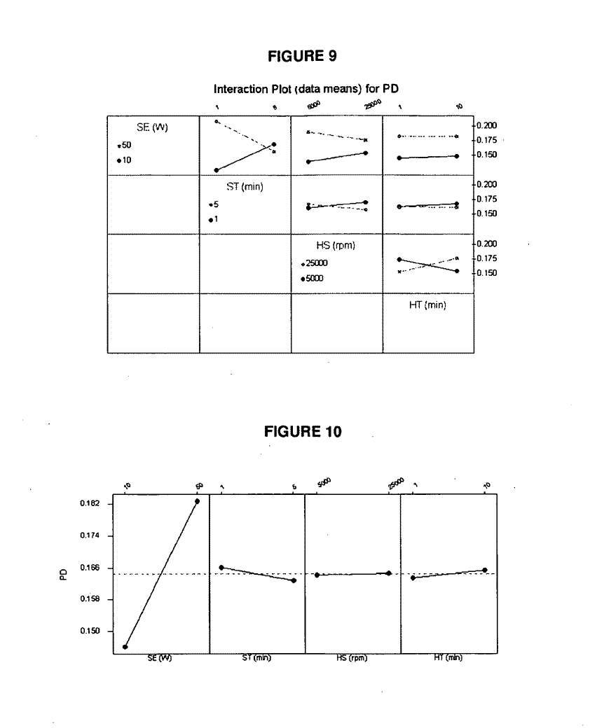

Polydispersity index is used to describe the spread in particle diameters for a given sample of particles. Values approaching zero in this method of particle size determination indicated a narrow size range (McCarron et al., Int. J. Pharm. 193, 37-47 1999). From FIG. 9, it is evident that sonication energy is the single most important factor in controlling polydispersity. A similar dependence of polydispersity on sonication energy was observed by Pereira-Lachataignerais et al. Chem. Phys. Lipids. 140, 88-97 (2006). From FIG. 10, it can be seen that there was a significant (P<0.05) interaction between sonication energy and sonication time in influencing polydispersity.

Sonication time was further increased from its high level of five minutes in Experiment 6 to eight minutes (Experiment 18; Table 4), keeping all the other parameters constant. As predicted by the results of the factorial design, effective diameter decreased with increase in sonication energy (Table 4). Similarly, homogenization speed was increased from its high level of 25,000 rpm in Experiment 4 to 30,000 rpm (Experiment 19; Table 4). Increase in homogenization speed resulted in an increase in the effective diameter, as predicted by the model. These observations confirm the validity of the results derived using the factorial design study.

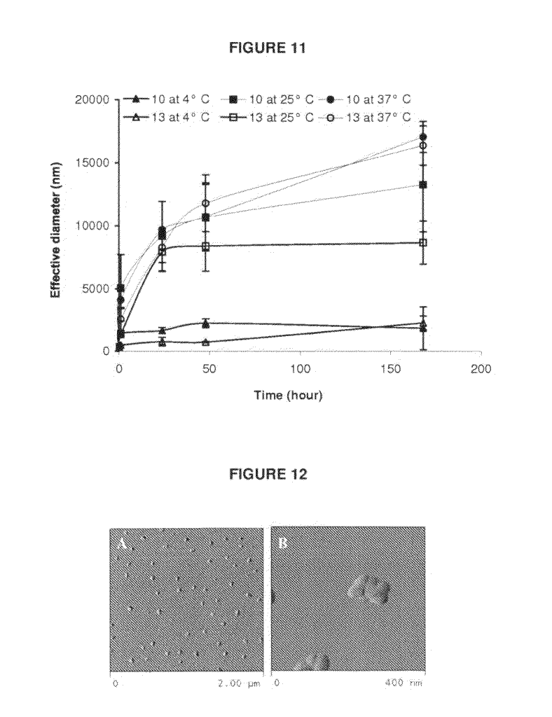

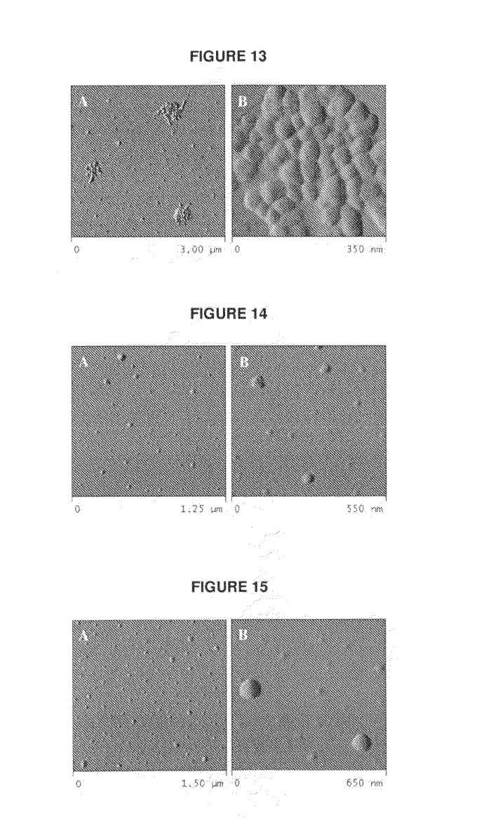

To evaluate the stability of nanoparticles, nanoparticle dispersions in buffer were stored at 4° C., 25° C., and 37° C., and changes in particle size and morphology were determined as a function of time. In general, it was observed that for both Experiments 10 and 13 formulations, nanoparticle size was higher at the end of one week (FIG. 11). The particle size increase was greater for nanoparticles stored at 25° C. and 37° C. than for those stored at 4° C. Without being bound by a mechanism, higher temperatures could have increased the kinetic energy in the system, resulting in greater number of collisions and the possibility of aggregation. The presence of aggregates was confirmed by AFM. AFM images of nanoparticles from Experiment 10 (FIGS. 12A and B) and Experiment 13 (FIGS. 13A and B) stored for 48 hours showed the presence of a larger number of aggregates for Experiment 13. Nanoparticles in Experiment 13 were formulated using higher sonication energy, and this could have imparted greater kinetic energy to the system, resulting in the formation of larger aggregates. No or minimal aggregation was observed in nanoparticles from both Experiment 10 (FIGS. 14A and B) and Experiment 13 (FIGS. 15A and B) prior to storage.

In this Example, fractional factorial design was used to evaluate the effect of various processing conditions on nanoparticle properties such as particle size, zeta potential, and polydispersity. The results indicate that sonication energy, sonication time, and homogenization speed were important determinants of nanoparticle size. Zeta potential was affected only marginally by any of the process parameters studied. Sonication energy was the biggest determinant of nanoparticle polydispersity. AFM studies suggest that although two different energy inputs may result in similar particle size, nanoparticles prepared with lower energy input were more stable against aggregation upon storage.

### Example 2

### Biodistribution of Drugs in Mice Treated with Drug-Loaded Nanoparticles

As disclosed in this Example, nanoparticles enhanced drug levels in brain. Experiments were conducted as follows. Nanoparticles containing brain-derived lipids were formulated containing either BMS 182874 (BMS) or 6-coumarin. BMS does not penetrate the BBB, and hence, was used as a model for poorly permeable drugs. 6-coumarin is a highly lipophilic fluorescent molecule that penetrates the BBB very well, and hence, was used a model for well permeated drugs.

BMS or 6-coumarin (5 mg), brain lipid (20 mg), lecithin (75 mg), and stearic acid (100 mg) were dissolved in 20 mL chloroform and the chloroform was evaporated by using nitrogen gas evaporator, to produce a clear lipid phase. A pre-dispersion of the clear lipid in the aqueous phase (2.5 mL distilled water) was prepared by magnetic stirring for 10 min. This pre-mix was passed through a high-pressure homogenizer for five cycles at 10,000 rpm for five minutes. All the nanoparticle formulations had a mean diameter in the size range of 179±48 nm as measured by dynamic light scattering. Encapsulation efficiency of the drug in the formulation was found to be 72.0±0.8%.

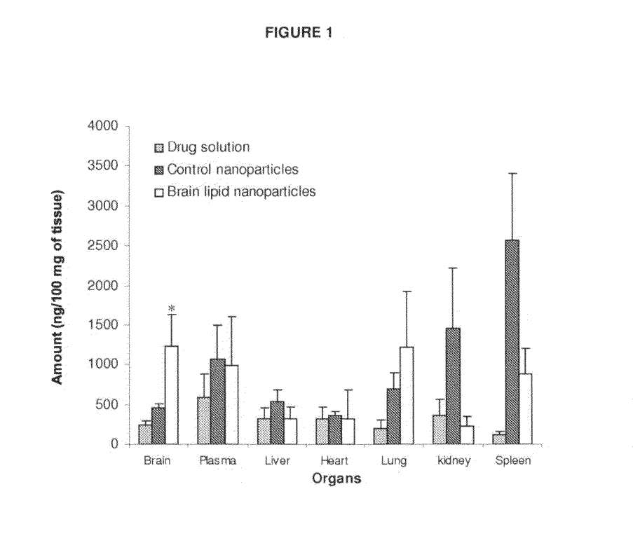

The brain delivery of BMS was assessed one hour following intravenous administration of brain lipid-coated nanoparticles in mice (FIG. 1). BMS in nanoparticles with or without brain-derived lipids or equivalent amount in solution (12 mg/kg) was injected intravenously in mice through tail vein, and drug levels in different tissues at the end of 1 hr was analyzed by HPLC. Data as mean±SD (n=6). * p<0.05.

Encapsulation of the drug in brain lipid-coated nanoparticles resulted in a 5-fold increase in drug levels in the brain compared to solution control (236±58 ng/100 mg of tissue Vs1224±413 ng/100 mg of tissue; p<0.05). Encapsulation of drug in uncoated nanoparticles also resulted in a moderate but significant (p<0.05) increase in drug levels in brain (458±47 ng/100 mg of tissue) compared to solution control. The results thus suggest that brain lipids enhance the delivery of nanoparticle encapsulated drug to the brain.

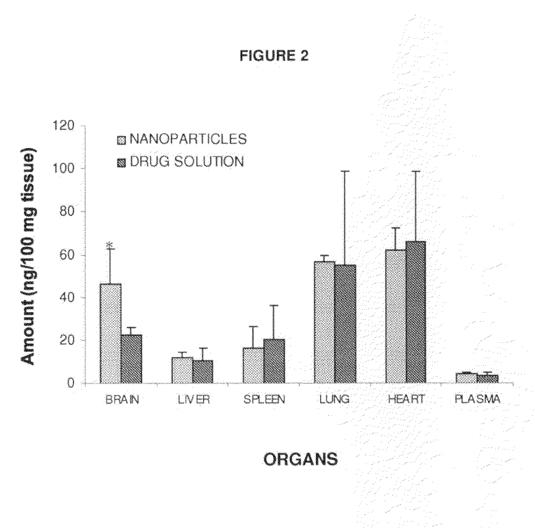

Similar results were obtained with 6-coumarin, a dye that penetrates BBB well. Nanoparticles enhanced the brain delivery of 6-coumarin about 2.1-fold (p<0.05) compared to that with the drug in solution (FIG. 2). 6-coumarin in nanoparticles or equivalent amount in solution was injected intravenously in mice through tail vein and drug levels in different tissues at the end of 1 hr was analyzed by HPLC. Data as mean±SD (n=4 and 3). * p<0.05.

These results confirm that nanoparticles comprising brain-derived lipids enhanced the brain delivery of encapsulated drugs.

While the description above refers to particular embodiments of the present invention, it will be understood that many modifications may be made without departing from the spirit thereof. The presently disclosed embodiments are therefore to be considered in all respects as illustrative and not restrictive. All publications referenced herein are hereby incorporated by reference.

What is claimed is:

1. A nanoparticle composition comprising:
a) a brain lipid,
b) a supplemental lipid,
c) a PEG-conjugated lipid, and
d) a drug or therapeutic compound.

2. The composition of claim 1 wherein the brain lipid is a phospholipid.

3. The composition of claim 1 wherein the brain lipid is selected from the group consisting of phosphatidylethanolamine, phosphatidylserine, phosphatidylcholine, phosphatidic acid, phosphatidylinositol, and combinations thereof.

4. The composition of claim 1 wherein the supplemental lipid is a long-chain fatty acid.

5. The composition of claim 1 wherein the supplemental lipid is selected from the group consisting of stearic acid, linolic acid, linoleic acid, and palmitic acid, and combinations thereof.

6. The composition of claim 1 wherein the supplemental lipid is stearic acid.

7. The composition of claim 1 wherein the PEG-conjugated lipid is distearoylphosphatidylethanolamine-PEG.

8. The composition of claim 1 wherein the drug or therapeutic compound is targeted toward the central nervous system.

9. The composition of claim 1 wherein the drug or therapeutic compound is selected from the group consisting of hydrophobic drugs, anti-cancer drugs, drugs for the treatment of epilepsy, drugs for the treatment of brain injury, drugs for the treatment of amylotrophic lateral sclerosis, drugs for the treatment of bacterial infections, and drugs for the treatment of viral infections, and combinations thereof.

10. The composition of claim 1, wherein the average diameter of the nanoparticles in the composition is between 10 and 1000 nanometers.

11. The composition of claim 1, wherein the average diameter of the nanoparticles in the composition is between 30 and 500 nanometers.

12. The composition of claim 6, wherein the average diameter of the nanoparticles in the composition is between 50 and 350 nanometers.

13. A method of delivering drugs or therapeutic compounds to the brain of an individual comprising:
a) providing a nanoparticle composition, and
b) administering the nanoparticle composition to an individual, wherein at least a portion of the therapeutic composition is delivered to the brain.

14. The method of claim 13 wherein the nanoparticle composition comprises:
a) a brain lipid,
b) a supplemental lipid,
c) a PEG-conjugated lipid, and
d) a drug or therapeutic compound.

15. The method of claim 13 wherein the nanoparticle composition is administered to an individual by intravenous administration.

16. The method of claim 13 wherein the nanoparticle composition is administered to an individual by intraperitoneal administration.

17. The method of claim 13, wherein the average diameter of the nanoparticles in the composition is between 10 and 1000 nanometers.

18. The method of claim 13, wherein the average diameter of the nanoparticles in the composition is between 30 and 500 nanometers.

19. The method of claim 13, wherein the average diameter of the nanoparticles in the composition is between 50 and 350 nanometers.
